(19) United States
(12) Patent Application Publication
(10) Pub. No.: US 2026/0345199 A1
(43) Pub. Date: Nov. 5, 2026

(54) CRANIAL ACCESS PORTS

(71) Applicant: Icahn School of Medicine at Mount Sinai, New York, NY (US)

(72) Inventors: Isabella C. MORGAN, New York, NY (US); Turner S. BAKER, New York, NY (US); Alexis Angelo Jacques BRUHAT, New York, NY (US); Benjamin RAPOPORT, New York, NY (US); Joseph BORRELLO, New York, NY (US)

(73) Assignee: Icahn School of Medicine at Mount Sinai, New York, NY (US)

(21) Appl. No.: 19/487,591

(22) PCT Filed: May 30, 2024
(86) PCT No.: PCT/US2024/031580

§ 371 Date: Nov. 25, 2025

Related U.S. Application Data
(60) Provisional application No. 63/469,882, filed on May 31, 2023.

(51) Int. Cl. A61B 17/34; A61B 1/313; A61B 17/00

(52) U.S. Cl. CPC A61B 17/3423; A61B 1/313; A61B 2017/00238; A61B 2017/3445; A61B 2017/3492

(57) ABSTRACT

A cranial access port includes a port body having a bend incorporated therein and having an ellipse cross-section. The port body is configured to be disposed at least partially within an subdural space for removal of a subdural hematoma and the port body has at least one lumen formed therein. The at least one lumen is configured to receive at least one tool for performing at least one operation within the subdural space. The at least one tool can be a visualization device, such as an endoscope, and at least one other tool. The cranial access port is configured to rotate 360 degrees within the subdural space such that in combination with the visualization device, the surgeon has real-time imaging of the subdural space.

### CROSS-REFERENCE TO RELATED PATENT APPLICATIONS

This application is based on and claims priority to U.S. Provisional Patent Application 63/469,882, filed May 31, 2023, the entire contents of which is incorporated by reference herein as if expressly set forth in its respective entirety herein.

### TECHNICAL FIELD

The present disclosure is directed to subdural hematoma evacuation devices and more particularly, to cranial access ports for use in a subdural hematoma evacuation procedure and more particularly, to cranial access ports that can be used in a minimally invasive procedure at the beside or in an angiography suite and are configured to permit delivery of one or more surgical tools to the subdural space, as well as providing a 360 degree real time view of the subdural space.

### BACKGROUND

A subdural hematoma (SDH) represents a blood collection, located between the dura and the middle layer of the meninges. This condition tends to have a compressive effect on the brain, which can lead to neurological effects and even death. When the blood is first collected, it has a viscous consistency. At this stage, the subdural hematoma is considered to be acute. As the hematoma progresses, the blood becomes thin, encapsulated, and liquified, at this stage, the hematoma is considered to be chronic (CSDH).

Subdural hematomas tend to develop after a traumatic event and are mostly produced due to falls from the same or another level. In a traumatic brain injury, the inertial movement that arises after the acceleration and deceleration of the skull and brain combo leads to a rupture of the bridging veins in the subdural space. Subdural hematomas are significantly more likely in older people. It is predicted given current trends, CSDH will surpass primary brain tumors and metastases as the most common cranial surgical condition in the U.S. Without treatment, a subdural hematoma can be fatal. Approximately one-third of all chronic subdural hematoma patients die and another one-third become permanently disabled.

If surgically removed, the hematoma capsule is usually reached and resolved with one of the two main treatments, a burr hole opening or a craniotomy. A craniotomy is generally reserved for an extensive hematoma and is considered a second-tier surgical option for most cases. It requires the surgeon to remove a relatively large piece of skull, evacuate the hematoma, and then reattach the flap back on the patient. A burr hole evacuation, in turn, is performed by suctioning and irrigating the blood out of the skull through a small hole; this procedure is generally preferred due to its low invasiveness.

However, compared to the craniotomy, a burr hole is not nearly as effective at evacuating the hematoma and is more likely to cause recurrence in patients. The recurrence occurs due to the small window the burr hole provides the surgeon with. Without complete access to the hematoma, the surgeon is rarely able to evacuate it properly, which leads to postoperative complications. Furthermore, clinical trials are currently ongoing to test the safety and efficacy of an up and coming technique called middle meningeal artery (MMA) embolization. MMA embolization is an intervention designed to occlude or restrict blood flow to these capillaries thereby preventing leakage. A downside to this technique, is that patients that undergo surgical evacuation and MMA embolization would need to receive two separate anesthesia sessions as one procedure is performed in the operating room, and the other in the angiography suite.

A third surgical option can be referred to as being a bedside procedure in that there are commercially available evacuation tools for subdural hematomas that are designed to be used at the bedside. These types of tools include equipment needed for burr hole drilling, insertion and blood drainage. If effective, bedside procedures are cheaper for both the hospital and the patient, less invasive, and faster. However, these commercially available tools fail to achieve the same effectiveness as a craniotomy or a burr hole evacuation. In particular, these types of tools tend to have a much higher likelihood to cause a recurrence than craniotomy.

It is also important to note that without any treatment, few chronic subdural hematomas have been reported to regress spontaneously, whereas 40% of all patients may recover without surgical intervention. However, 30% of patients undergoing conservative management require surgical intervention.

Accordingly, there is a clear need for an effective treatment option at the bedside and angiography suite that is effective, safe and decreases the probability of recurrence.

### SUMMARY

A cranial access port is disclosed and is configured to overcome the deficiencies noted above with respect to traditional subdural hematoma evacuations and provide an improved solution for subdural hematoma evacuations. As described herein, the cranial access ports disclosed herein provide a subdural hematoma evacuation solution that can be performed at bedside and in the angiography suite, thereby decreasing the number of anesthesia sessions necessary, which can be cumbersome for older patients. The present solution also provides active suction and a 360 degree view of the subdural space will provide the surgeon with the force necessary to remove viscous hematoma and the visual feedback to identify key structures and determine evacuation completion.

The cranial access port, according to one embodiment, comprises a port body having a bend incorporated therein and having an ellipse cross-section. The port body is configured to be disposed at least partially within a subdural space and the port body having at least one lumen formed therein. The at least one lumen is configured to receive at least one tool for performing at least one operation within the subdural space. The cranial access port is primarily discussed for use in the subdural space; however, other applications may be possible such as treatment of epidural and intraparenchymal hematoma.

In one embodiment, the at least one lumen comprises a first lumen and a second lumen and the at least one tool comprises a visualization device, such as an endoscope, and at least one other tool. The first or second lumen is configured to receive the endoscope and the other of the first or second lumen is configured to receive the at least one other tool. The visualization device, in combination with the 360 degree rotation, provides the surgeon with a 360 degree real time view of the subdural space.

The bend can be defined by an angle greater than 0 to 180 degrees, more particularly, by an angle greater than 0 to 90 degrees. For example, the bend can be a 90 degree bend.

The cranial access port also includes a stabilizing base that is configured to be fixedly attached to a patient's scalp and includes an opening to permit the port body to pass therethrough.

The at least one other tool comprises at least one tool selected from the group consisting of: an irrigation tool, an aspiration tool, a coagulation tool, a tissue grasping tool, or combinations thereof.

### BRIEF DESCRIPTION OF THE DRAWING FIGURES

FIG. 1 is a side and top perspective view of a cranial access port according to one embodiment;

FIG. 2 is a side elevation view thereof;

FIG. 3 is a front elevation view thereof;

FIG. 4 is a side perspective view of an access port body of the cranial access port of FIG. 1;

FIG. 5 is a perspective view of a stabilizing base for use with the cranial access port;

FIG. 6 is a top plan view;

FIG. 7 is a front elevation view of a cranial access port according to another embodiment;

FIG. 8 is a cross-sectional view taken along the line B-B in FIG. 7;

FIG. 9 is a cross-section view taken along the line A-A in FIG. 7;

FIG. 10 is a front elevation view of a cranial access port according to one embodiment;

FIG. 11 is a front elevation view of a cranial access port according to another embodiment;

FIG. 12 is a front elevation view of a cranial access port according to another embodiment;

FIG. 13 is a cross-sectional view taken along the line A-A of FIG. 12;

FIG. 14 is close-up view of a portion of FIG. 13;

FIG. 15 is a front elevation view of a cranial access port according to another embodiment;

FIG. 16 is a cross-sectional view taken along the line A-A of FIG. 15;

FIG. 17 is a front elevation view of a cranial access port according to one embodiment that utilizes a rigid endoscope and mirror;

FIG. 18 is a cross-sectional view taken along the line A-A of FIG. 17;

FIG. 19 is a close-up view of a portion of FIG. 18;

FIG. 20 is a front elevation view of a cranial access port according to one embodiment that utilizes a rigid endoscope and mirror;

FIG. 21 is a front elevation view of a cranial access port according to one embodiment that utilizes a flexible endoscope;

FIG. 22 is a cross-sectional view taken along the line A-A of FIG. 21;

FIG. 23 is a cross-sectional view taken along the line B-B of FIG. 21;

FIG. 24 is a front elevation view of a cranial access port according to one embodiment that utilizes a flexible endoscope;

FIG. 25 is a cross-sectional view taken along the line A-A of FIG. 24;

FIG. 26 is a front elevation view of a cranial access port according to one embodiment that utilizes a flexible endoscope;

FIG. 27 is a cross-sectional view taken along the line B-B of FIG. 26;

FIG. 28 is a front elevation view of a cranial access port according to one embodiment that utilizes a flexible endoscope;

FIG. 29 is a cross-sectional view taken along the line A-A of FIG. 28;

FIG. 30 is a cross-sectional view taken along the line B-B of FIG. 28;

FIG. 31 is a cross-sectional view of a patient's head showing a subdural hematoma;

FIG. 32 is a cross-sectional view of a patient's head showing the subdural hematoma with a cranial access port being passed through a burr hole; and

FIGS. 33A-33C illustrate the rotation of the cranial access port within the subdural space and applied suction for removing the subdural hematoma.

### DETAILED DESCRIPTION OF CERTAIN EMBODIMENTS

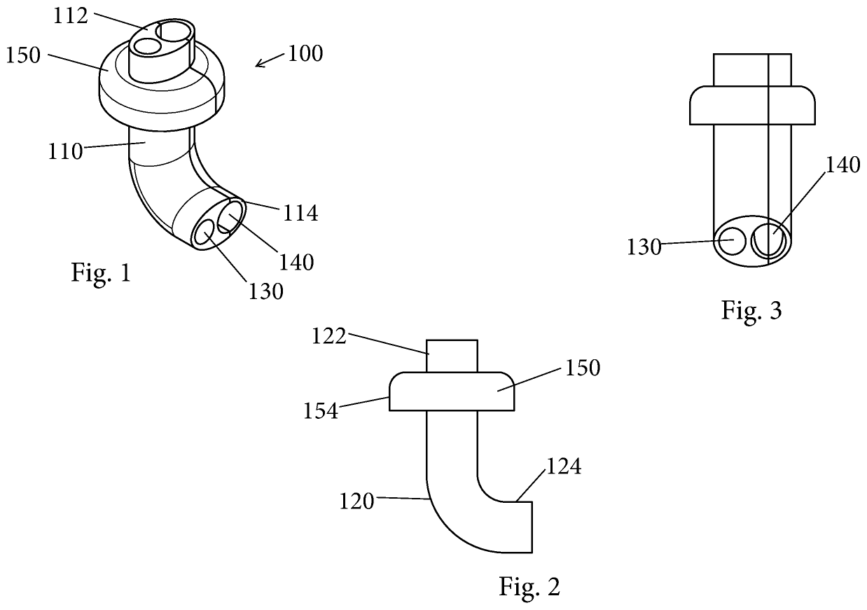

FIGS. 1-6 illustrate a cranial access port 100 that is constructed to overcome the deficiencies noted above and provide an improved solution for subdural hematoma evacuations. To successfully evacuate a subdural hematoma and provide an improved tool and surgical procedure, the evacuation tool must satisfy a number of requirements. Because subdural hematoma affects mainly older people, who tend to have a series of comorbidities, a minimally invasive evacuating surgery is highly beneficial. Additionally, creating a solution that can be performed at bedside and in the angiography suite will decrease the number of anesthesia sessions necessary, which can be cumbersome for older patients. Lastly, a solution that provides active suction and a 360 degree view of the subdural space will provide the surgeon with the force necessary to remove viscous hematoma and the visual feedback to identify key structures and determine evacuation completion.

It will be appreciated that any dimensions listed in the drawings are merely exemplary for one or more embodiments and not limiting of the present devices disclosed herein.

As understood by one skilled in the art, the subdural space (or subdural cavity) is a potential space that can be opened by the separation of the arachnoid mater from the dura mater as the result of trauma. The dimensions of the subdural space will vary from patient to patient as a result anatomical differences, etc. A subdural hematoma is thus a buildup of blood on the surface of the brain. The blood builds up in a space (subdural space) between the protective layers that surround the brain.

The cranial access port 100 is defined by an access port body 110 that has a first end 112 and an opposite second end 114. Each of the first end 112 and the second end 114 can have a flat, planar surface. As shown in the figures, the access port body 110 comprises a bent structure in that there is a bend formed along a length of the access port body 110. The bend defines a bent section 120 that is located between an upper section 122 and a lower section 124. The bend in the access port body 110 can be defined by an angle that is greater than 0 and is 180 degrees or less and in one embodiment, the bend is defined by an angle that is greater than 0 and is 90 degrees or less. As shown, the bent section 120 is not formed as a sharp bend but instead has soft curvature within the bent section 120. In other words, there is a soft curve from the lower section 124 to the upper section 122.

In the illustrated embodiment, the angle of the bend is 90 degrees and therefore, a longitudinal axis of the upper section 122 that passes through a center of the upper section 122 at the first end 112 is perpendicular to a longitudinal axis of the lower section 124 that passes through a center of the lower section 124 at the second end 114. This results in the flat, planar surface at the first end 112 lying in a plane that is perpendicular to a plane that contains the flat, planar surface at the second end 114. The lengths of the upper section 122 and lower section 124 can be different, as shown, and more particularly, the upper section 122 can have a greater length than the lower section 124.

In view of the intended placement of the cranial access port 100 into the subdural space, the access port body 110 is configured for placement in such subdural space and therefore, the access port body 110 has a complementary cross-sectional shape for insertion and placement within the subdural space. More particularly, the access port body 110 has an ellipse cross section as shown. The ellipse cross-sectional shape is defined by a major axis that extends end-to-end of the access port body 110 and a minor axis that extends side-to-side of the access port body 110 and is oriented perpendicular to the major axis. The lengths of the major axis and the minor axis permit the access port body 110 to be received within the subdural space. For example, the lengths of the major axis and the minor axis can be selected so that the access port body 110 can be received within a 1 cm sized subdural space. In one embodiment, the length of the major axis can be at least 8 mm and the length of the minor axis is less than 8 mm and more particularly, the length of the minor axis can be from 4 mm to 6 mm. It will be appreciated that the aforementioned dimensions are merely exemplary in nature and are not limiting of the scope of the cranial access ports described herein.

As illustrated, the ellipse cross-section shape is uniform from the first end 112 to the second end 114.

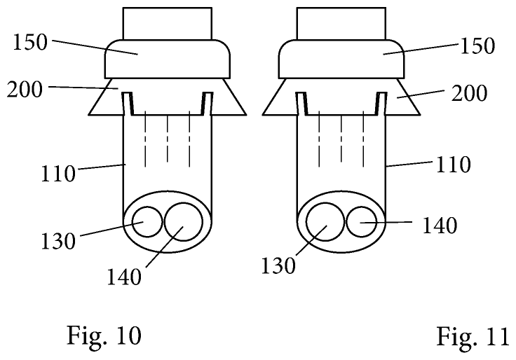

The access port body 110 has at least one lumen formed therein each of which is open at both the first end 112 and the second end 114. In the embodiment shown in FIGS. 1-6, the access port body 110 includes a first lumen 130 and a second lumen 140. As mentioned, each of the first lumen 130 and the second lumen 140 is open at the first end 112 and the second end 114 and they remain separated from one another along their complete lengths. The first lumen 130 and the second lumen 140 can be formed side-by-side with a separating wall therebetween. Any number of different sizes and shapes can be selected for the first lumen 130 and the second lumen 140. In the illustrated embodiment, each of the first lumen 130 and second lumen 140 has a circular shape with the diameter of one lumen being different than the other. For example, in FIGS. 1 and 10, the second lumen 140 has a greater diameter, while in FIG. 11, the first lumen 130 has the greater diameter. In another embodiment, described below, the diameters can be equal.

The number, shape and sizes of each lumen are selected in view of the one or more surgical tools that are inserted through the lumen to reach the subdural space and reach the subdural hematoma for evacuation thereof.

The cranial access port 100 also includes a collar (flange member) 150 that surrounds the upper section 122 of the access port body 110 above the bent section 120. The collar 150 is not at the first end 112 but is rather offset therefrom and therefore, there is a first portion of the upper section 112 that lies above the collar 150 and is freely accessible to the user. A second portion of the upper section 112 lies below the collar, with, in the illustrated embodiment, the length of the first portion being significantly less than the length of the second portion. The collar 150 extends radially outward from the access port body 110 and can have a different shape than the access port body 110. As illustrated, the collar 150 has a circular shape.

In one embodiment, both the collar 150 and access port body 110 are formed of a suitable plastic material. The collar 150 can therefore be integrally formed with the access port body 110 as, for example, part of a common molding process or the collar 150 can be attached to the access port body 110 in surrounding fashion (e.g., using bonding techniques). The collar 150 thus does not move relative to the access port body 110. As shown in the cross-sectional view of FIGS. 8 and 9, the collar 150 has a hollow underside that is defined by a peripheral side wall 152 that defines a hollow interior space 154. The peripheral side wall 152 has an annular shape and the hollow interior space has a first height (H1).

### Single Lumen Construction

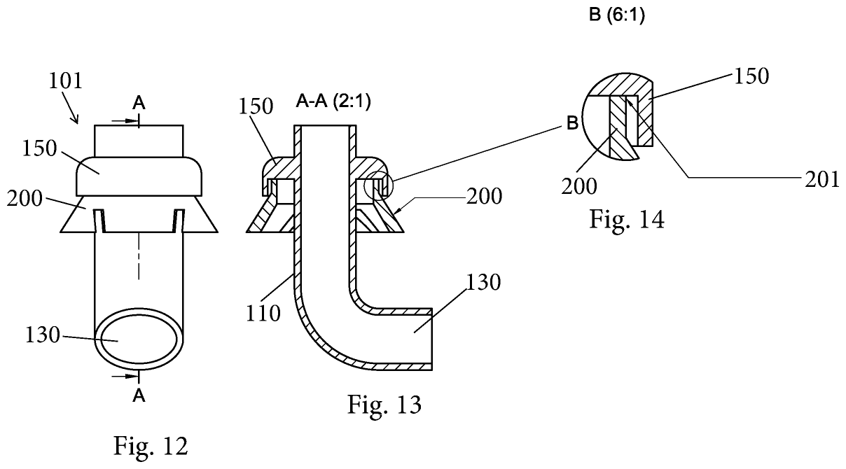

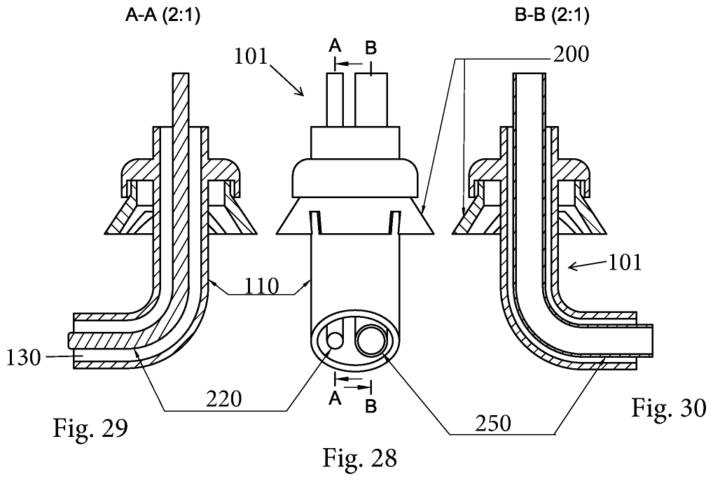

The embodiment of FIGS. 12-14 is directed to a cranial access port 101 that includes only a single lumen (i.e., first lumen 130). The cranial access port 101 can otherwise be the same as the cranial access port 100 and therefore, like elements are numbered alike. In the single lumen design, each of the surgical tools (e.g., visualization and evacuation tools) are inserted/delivered through the single lumen as shown in FIGS. 28-30.

### Retractor (Stabilizing) Base

The cranial access port 100 can further include a retractor base 200 which is separate and removable from the combined access port body 110 and the collar 150. More particularly, the retractor base 200 is configured to engage the cranial access port 100 during the surgical procedure as discussed herein.

The retractor base 200 has a top end 202 and an opposite bottom end 204. The retractor base 200 has an upper section 210 and a bottom section 220. The upper section 210 is configured to mate with and detachably couple to the collar 150. The upper section 210 has a complementary shape to the collar 150 and thus, the upper section 210 can have an annular shape with an outer diameter less than an inner diameter of the collar 150. The upper section 210 is intended to be received within the hollow interior space 154 and be securely coupled to the collar 150 as by a friction fit or the like. The coupling before the retractor base 200 and the collar 150 is of a type that the combined access port body 110 and the collar 150 can freely rotate about the retractor base 200. In FIG. 14, reference character 201 points to a mated interface (fit) between the collar 150 and the retractor base 200 to permit rotation therebetween.

The retractor base 200 has an outwardly flared lower section 220 that is integral to the upper section 210. The lower section 220 can thus be characterized as having a frustoconical shape. The lower section 220 has a plurality of open notches (slots) 230 formed therein. The plurality of open notches 230 can include a first pair of notches and a second pair of notches. The first pair of notches are formed along one half of the lower section 220 and the second pair of notches are formed along the other half of the lower section 220. One notch of the first pair can be generally located 180 degrees from one notch of the second pair. Each of the notches 230 is open along the bottom end 204.

The retractor base 200 is thus hollow and a through hole passing from the first end 202 to the bottom end 204.

In use, the plurality of notches 230 of the retractor base 200 are configured for receiving a scalp retractor tool. As described in more detail below, in use, the retractor base 200 is intended to be fixedly anchored to the scalp as by using one or more scalp retractor tools; however, the cranial access port 100 (i.e., the combined access port body 110 and the collar 150) can freely move (rotate) relative to the retractor base 200. In use, the scalp retractor tool enters into the notches 230 to anchor the retractor base 200 in place along the scalp.

The retractor base 200 can be characterized as being an external stabilizing structure. It will also be understood that other stabilizing structures can be used so long as they can be fixed to the scalp and permit free rotation of the cranial access body 110. For example, a tri-pod structure can be provided as the stabilizing structure.

In addition, the stabilizing structure can contain a drainage mechanism, such as a number of holes to support drainage.

### Intraluminal Tools

As mentioned, the cranial access ports disclosed herein have one or more lumens and in the case of the cranial access port 100, it has side-by-side first and second lumens 130, 140. These two lumens 130, 140 are designed to receive one or more intraluminal tool for use in the surgical procedure (i.e., the subdural hematoma evacuation).

### Visualization Device

One of the intraluminal tools can be in the form of a visualization device that allows the surgeon a 360 degree view of the subdural space, and in particular, a view of the blood tissue mass to be evacuated. Direct visualization of the subdural space enables qualification of the percent of the hematoma that has been evacuated. Identification of septations and avoidance of structures that may cause secondary bleeds when tampered. The inclusion of visualization in the cranial access port 100 allows for increased evacuation rates and decreases the likelihood of excess surgical trauma that is undergone in more invasive procedures. In other words, inclusion of visualization in the cranial access port 100 allows for an evacuation procedure that provides the benefits of a craniotomy in a minimally invasive manner.

It will be appreciated that any number of different visualization devices can be used as part of the cranial access port 100. For example, the visualization can be achieved through the use of a camera system embedded at the top of the cranial access port; a rigid endoscope; or a flexible endoscope. Each of these devices is discussed below.

It will be appreciated that the visualization device can be designed to be received within one of the lumens, for example, the first lumen 130 or the second lumen 140. As such, the figures included herewith illustrate the visualization device contained within the first lumen 130 and alternatively, contained the second lumen 140.

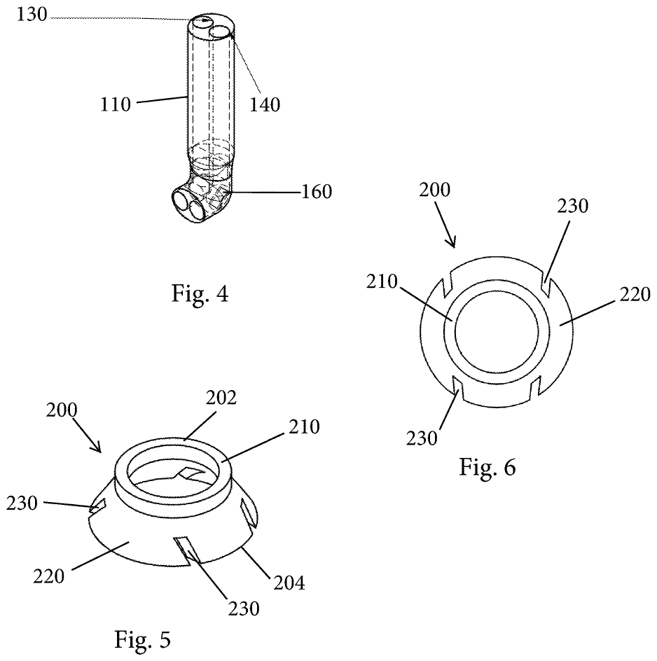

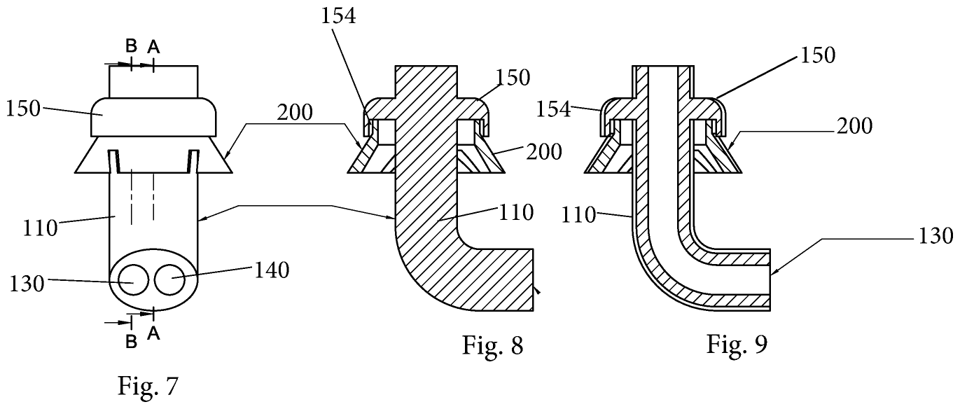

In the event that a camera system is used, the camera system is in communication with one of the first lumen 130 and the second lumen 140 which acts as a visualization lumen for visualization of the subdural space. The camera system is of a type suitable for inclusion in the cranial access port 100 and suitable for using the selected lumen 130, 140 as a visualization pathway for visualizing the subdural space. The camera system can be a digital camera, such as a digital CCD camera. Other types of cameras are equally possible as well. In the event that the camera system is located at or near the top (first end 112) of the access port body 110, a mirror 160 (FIG. 4) that is located within one lumen, in this case the second lumen 140. More particularly, the mirror 160 is located within the bent section 120 of the access port body 110 and is positioned such that light traveling within the linear upper section 122 of the access port body 110 is reflected by the mirror 160 into the lower section 124 of the access port body 110. The mirror 160 can be disposed at a 45 degree angle within the second lumen 140 so as to reflect light at an angle of 90 degrees into the lower section 124.

The camera device is thus located above the mirror 160 but the angle of the mirror 160 allows for the camera device to image and visualize the subdural space. As a result, the camera device does not physically enter the subdural space and is in fact not located in the lower section 124. The distal end of the camera device can be located within the upper section 122 or can enter into the bent section 120.

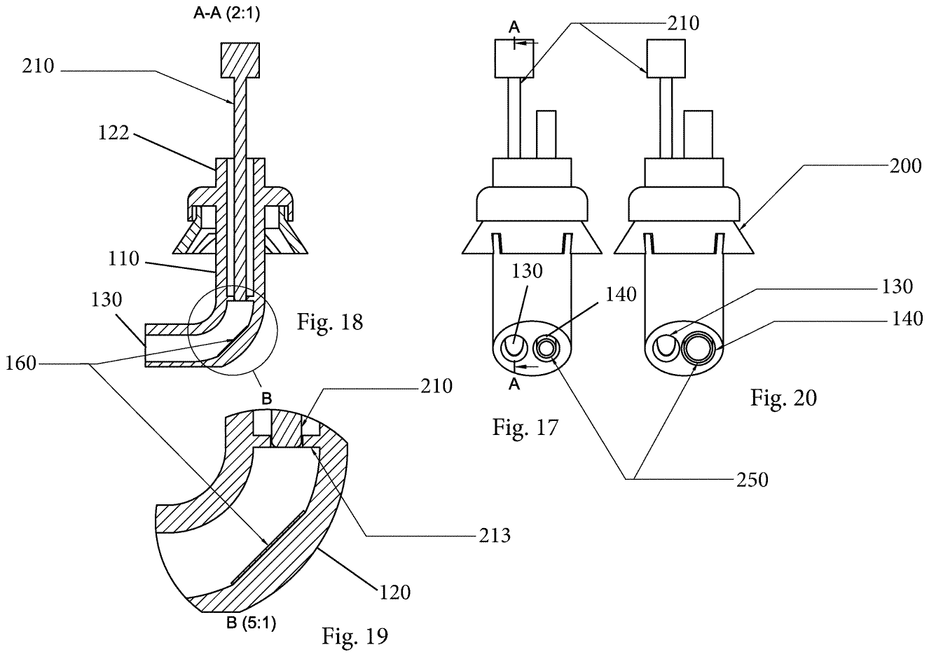

As mentioned, alternatively, the visualization device can be in the form of a rigid endoscope 210 as shown in FIGS. 17-20. Similar to the camera device described above, the rigid endoscope is disposed within the upper section 122 or can extend into the bent section 120 of the access port body 110 above the mirror 160. As known, a rigid endoscope is one which cannot be bent to go around corners but instead has a fixed linear configuration. Much like the camera device, the rigid endoscope 210 utilizes the mirror 160 to visualize the subdural space. As shown in FIG. 19, the distal end of the rigid endoscope 210 can be located within the bent section 120 or it can terminate within the upper section 122.

The rigid endoscope 210 is thus inserted at least partially within one of the first lumen 130 and the second lumen 140. The access port body 110 can have an endoscope docking feature in that the construction of the endoscope and the access port body 110 are complementary and there can be a locating feature that ensures the endoscope is positioned at the desired location. In other words, not only does the docking feature ensure that the rigid endoscope is securely attached to the access port body 110 but also it ensures that the distal end of the rigid endoscope remains a desired distance from the mirror 160. This ensures optimal imaging occurs using mirror 160. The locating feature can be in the form of an inner stop formed within the selected lumen 130, 140 whereupon when the rigid endoscope is inserted into the lumen 130, 140, the rigid endoscope contacts the inner stop which prevents additional travel of the rigid endoscope within the lumen 130, 140 in the direction toward the mirror 160. The locating feature can also be of a keyed construction in that the lumen 130, 140 can include male or female features that mate with the opposite female or male features formed as part of the rigid endoscope. For example, along a length of the upper section 122, there can be one or more groove that receives one or more tabs that protrude outwardly from the body of the rigid endoscope. Reception of the one or more tabs within the one or more grooves locates and securely couples the rigid endoscope to the access port body 110. In addition, the bottom end of each groove can define the stop. The opposite is true in that is possible to construct the lumen 130, 140 to include one or more tabs that are received in one or more grooves formed in the body of the rigid endoscope. Other types of arrangements are equally possible.

In FIG. 19, a stop 213 is shown and is represented by an inner wall that extends partially across the lumen. This stop 213 has an opening (e.g., center opening) that aligns with the vision component of the endoscope; however, the opening is sized so that the outer diameter of the endoscope cannot pass through and thus when inserted, the endoscope abuts this inner wall (stop 213) and that limits the degree of travel in the lumen (but does not adversely impact the visualization of the endoscope). The center opening can be tapered.

FIG. 20 shows an embodiment in which the first lumen 130 is smaller than the second lumen 140. In FIGS. 17-20, the rigid endoscope 210 is located within the first lumen 130.

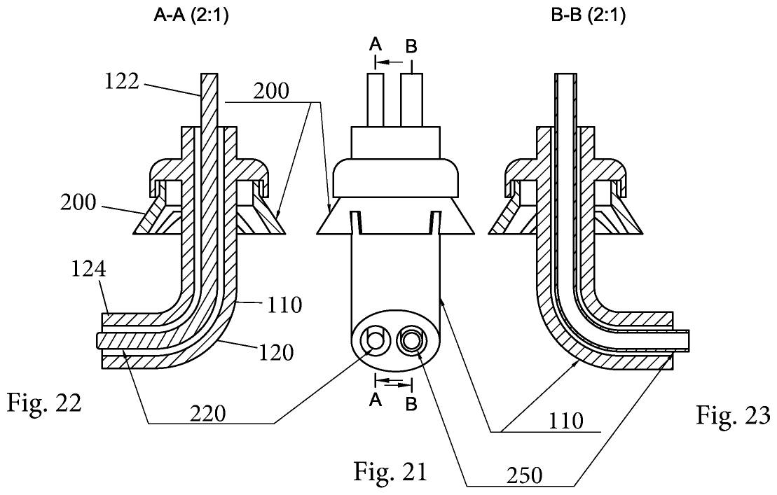

In another embodiment, the endoscope comprises a flexible endoscope 220 (FIGS. 21-27) that is inserted into one of the first lumen 130 and the second lumen 140. However, unlike the previous embodiments, the flexible endoscope 220 by nature is capable of being bent to travel around corners. Thus, the mirror 160 is eliminated in this embodiment. The flexible endoscope 220 is inserted into the respective lumen 130, 140 and is manipulated so that it travels through the bent section 120 to the lower section 124. The flexible endoscope 220 can be positioned at the open end of the lower section 124 or can extend beyond the lower section 124 such that at least a distal end of the flexible endoscope 220 is external to the lumen 130, 140 as shown.

It will also be appreciated that a steering mechanism can be employed to maneuver the flexible endoscope 220.

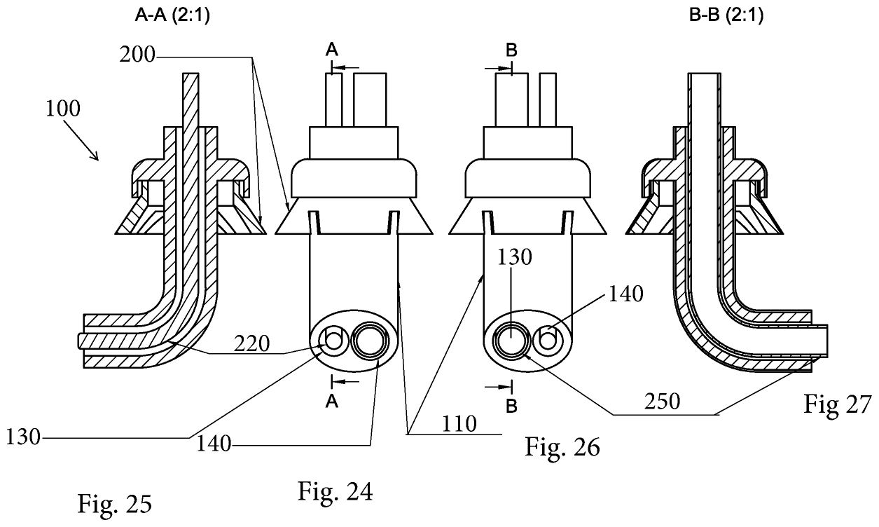

FIGS. 24-27 illustrate other embodiments that include the flexible endoscope 220. For example, in FIG. 24, the flexible endoscope 220 is located in the smaller first lumen 130, while in FIG. 26, the flexible endoscope 220 is located in the smaller second lumen 140.

As shown in FIGS. 28-30, when only a single lumen (first lumen 130) is used, the visualization device (e.g., flexible endoscope 220) is disposed in the first lumen 130 alongside other tools as explained below.

In the case of the camera device and the rigid endoscope, the subdural space is located perpendicular to the camera lens.

The visualization device (e.g., camera device, rigid endoscope, or flexible endoscope) is operatively coupled to a display to allow direct real time visualization of the subdural space as the surgical procedure (hematoma evacuation) is performed. The display can be part of a computing device that executes software to allow the display and recording of the visualization images. In yet another aspect, the visualization device can be operatively coupled to a computing device having a display in a wireless manner. In other words, the visualization device can include a communications module that is configured to transmit in real time the images of the visualization device to a remote computing device. The remote computing device can take many different forms including a laptop, desktop, tablet, etc. Any number of suitable communications protocols, including Bluetooth, can be used. In addition, the visualization device can be connected to the computing device by means of a cable.

### Other Surgical Tools

In the two lumen design shown in FIGS. 17-25, one lumen, such as the second lumen 140, is intended to receive one or more other surgical tools. The second lumen 140 can be considered to be a tool lumen intended to receive one or more tools other than a visualization device. These tools are intended to perform the hematoma evacuation. Alternatively, in FIGS. 26-27, the surgical tool is received within the first lumen 130 and the visualization device is in the second lumen 140.

For example, the at least one tool can be selected from the group consisting of: an irrigation tool, an aspiration tool, a coagulation tool, and a tissue grasping tool.

As known, an irrigation tool (instrument) is designed to deliver an irrigation solution to a target location, in this case the subdural space. Irrigation is a vital component of subdural hematoma evacuations. The constant introduction of saline solution into the subdural space allows for the resolution of clots and ultimate removal of the subdural hematoma.

The irrigation tool thus includes a lumen through which the irrigation solution is delivered. The irrigation tool is formed of a flexible body to allow the irrigation tool to bend and pass along the bent section 120 and then exit the bottom end of the access port body 110 into the subdural space. However, it will be appreciated that the distal end of the irrigation tool does not have to extend beyond the bottom end of the access port body 110 to deliver the irrigation fluid. The irrigation tool is fluidly connected to a source of irrigation fluid and a fluid management system, such as a pump, can be used to controllably deliver the irrigation fluid.

The irrigation tool can thus have a tubular structure and can be part of an irrigation mechanism in which irrigation is provided through syringe infusion or it can be part of an irrigation mechanism in which irrigation is powered by a pump system.

An aspiration tool is designed to remove (evacuate) debris and fluid from the surgical site (i.e., the subdural space). In the case of evacuation of a subdural hematoma, the hematoma is gently removed using aspiration (suction) and irrigation, where it's washed away with the irrigation fluid (saline solution). The aspiration tool is thus a tubular structure and is operatively connected to a suction source. The aspiration tool is formed of a flexible body to allow the aspiration tool to bend and pass along the bent section 120 and then exit the bottom end of the access port body 110 into the subdural space.

The aspiration tool can be part of a suction mechanism that is powered by wall suction or alternatively, the suction mechanism can be powered by a pump system.

As shown in FIGS. 17-27, the surgical tool is illustrated as a suction/irrigation catheter 250 that has a center lumen through which irrigation fluid can flow to the surgical site when the catheter 250 functions as an irrigation catheter 250 and catheter 250 can be operatively connected to suction when acting as an aspiration catheter.

It will also be appreciated that the irrigation tool and the aspiration tool can be combined into a single tool as opposed to using two tools that are fed through the one lumen 130, 140.

In addition, a coagulation tool can be used as part of the surgical procedure to remove the subdural hematoma.

A combined irrigation-coagulation suction cannula can be simultaneously used for irrigation and monopolar coagulation at its tip.

The at least one tool can also be in the form of a tissue grasping tool. As the name suggests, a tissue grasping tool is configured to grasp tissue at the surgical site. The tissue grasping tool is thus a flexible tool and can be advanced through the first lumen 130 and can be remotely controlled to allow the working distal end of the tissue grasping tool to grasp tissue at the surgical site.

### Steering Mechanism

The cranial access port 100 can also include a steering mechanism that is designed for and enables 360 degree rotation of the cranial access port 100. As discussed above, the access port body 110 is rotated 360 degrees relative to the retractor base 200 (stabilizing base) to allow for 360 degree visualization of the subdural space.

It will be appreciated that the steering mechanism can be of a manual type or of an automated (motorized) type. The steering mechanism is thus configured to provide controlled rotation of the access port body 110 to allow the surgeon to have complete visualization of the subdural space and allow assessment of the hematoma evacuation in real-time. Complete visualization of the subdural space allows the surgeon to assess the progress of the hematoma evacuation and to make sure that a complete evacuation of the hematoma is accomplished.

### Subdural Hematoma Evacuation Procedure

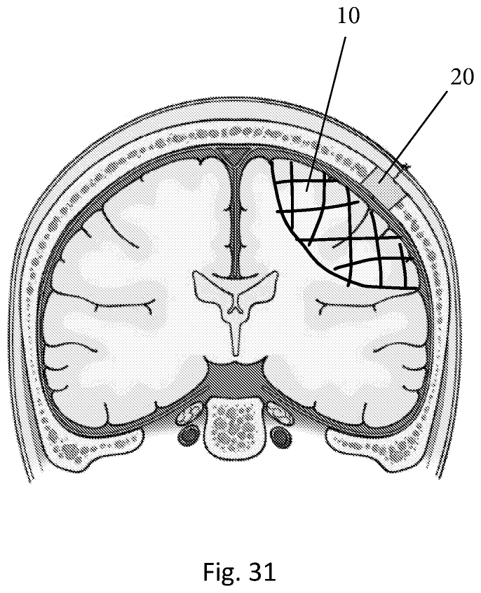

Now referring to FIGS. 31-33C in which a subdural hematoma evacuation is illustrated.

FIG. 31 is a cranial cross-section that illustrates a subdural hematoma 10 that is to be evacuated.

FIG. 31 also illustrates formation of a burr hole 20 through the skull to access the subdural hematoma 10. The burr hole 20 is thus formed at a location that permits access to and evacuation of the subdural hematoma.

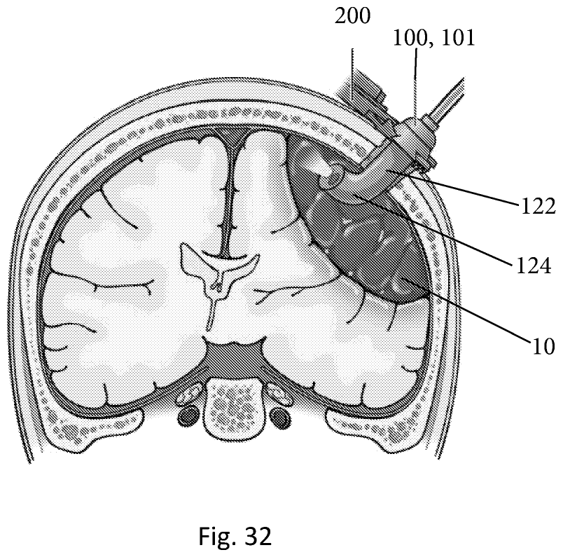

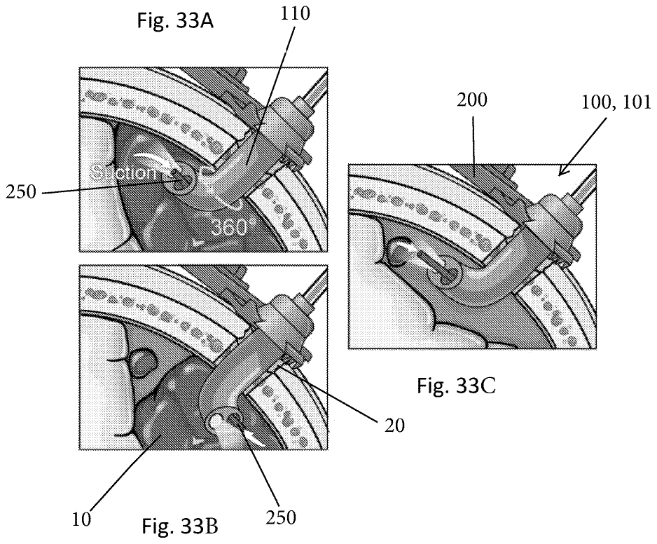

As shown in FIG. 32, after formation of the burr hole 20, the cranial access port 100, 101 is inserted into the burr hole and manipulated so as to position the lower section 124 in the subdural space. Due to the bent nature of the cranial access port 100, 101 the cranial access port 100, 101 is hooked through the burr hole 20 to position the lower section 124 in the subdural space. As shown, the upper section 122 is positioned perpendicular to the skull, while the axis of visualization is perpendicular to the upper section 122. The cranial access port 100, 101 is fixed to the skull using the retractor base 200 in the manner described herein. Due to the rotation of the cranial access body 110 relative to the retractor base 200, visualization of the subdural space is performed by rotating the cranial access body 110 over 360 degrees (See, FIGS. 33A-33C). Such controlled visualization permits the surgeon to evacuate the hematoma under visualization. Since the irrigation and suction is performed through one lumen of the access port body, the irrigation and suction is located along the center axis of the lower section 124. Thus, rotation of the cranial access port not only changes the imaging axis (field of view) but also changes the direction (axis) of the irrigation and suction. This allows the surgeon to be guided by the real time feedback of the visualization device which allows the surgeon to move not only the cranial access s port but also the intraluminal tools (suction and irrigation, etc.) to locations at which the subdural hematoma is present as part of the complete evacuation thereof.

As mentioned, irrigation is a vital component of subdural hematoma evacuations. The constant introduction of saline introduction into the subdural space allows for the resolution of clots and ultimate removal of the subdural hematoma. The active suction component allows for the removal of the saline hematoma combination. After the hematoma is resolved, a drain is placed either subdurally or extradurally to allows for the remaining saline and hematoma to flow out of the skull. This process is achieved naturally, as the body produces CSF and repopulates the area.

FIGS. 33A-33C illustrate the aspiration process and rotation of the cranial access port 100, 101 to ensure that the subdural hematoma 10 is completely removed from the surgical site. The visualization feature of the cranial access port 100, 101 is shown graphically in FIGS. 33A-33C. In addition, suction applied to the catheter 250 is shown.

One the subdural hematoma evacuation is completed, the surgical tools are removed from the lumens and the cranial access port is decoupled from the stabilizing base. Once completely removed, the burr hole is closed in a conventional manner.

### Bedside Application

In one aspect, the cranial access port 100, 101 comprises a subdural hematoma evacuation system whose intended use is at the bedside to allow for a fast and minimally invasive treatment. This allows the care of patients without taking them to the operating room. This is highly beneficial for both the patient and the hospital. A bedside solution decreases hospital costs by providing care without the need to use the operating room, which requires expensive equipment, procedures and extra personnel. For the patient, a bedside solution is highly beneficial as it is less invasive than a surgery in the OR and doesn't require the use of general anesthesia.

### Angiography Suite Application

In one aspect, the cranial access port 100, 101 comprises a subdural hematoma evacuation system whose additional intended use is in the angiography suite to allow for a fast and minimally evacuation to be performed in the same setting as MMA embolization. Currently, surgical evacuations and MMA embolization are performed in two different settings. The evacuation is generally performed in the operating room and the MMA embolization in the angiography suite, which requires the patient to undergo two sessions of anesthesia. The present solution that allows for evacuations to take place in the angiography suite would allow for both treatments to be done in a single combined procedure, with only one session of anesthesia. This is highly beneficial for the hospital and the patient, as it would decrease hospital costs and the invasiveness of both combined procedures.

### Flexible Endoscope With Working Channels

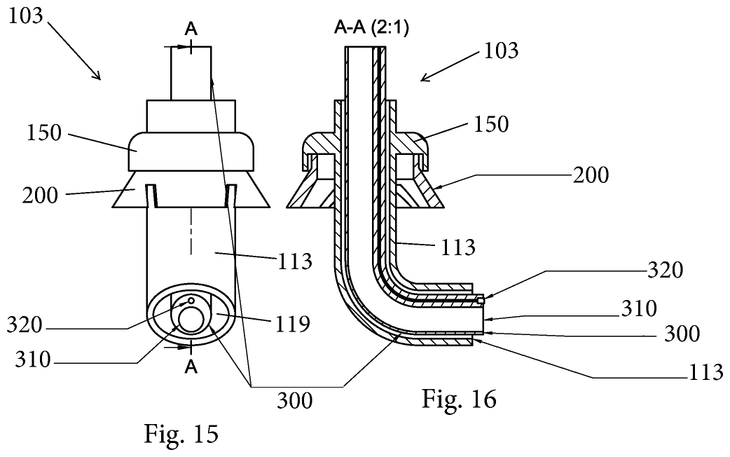

Now referring to FIGS. 15-16 in which a cranial access port 103 according to another embodiment is illustrated. The cranial access port 103 is similar to the other cranial access ports disclosed herein and therefore, like elements are numbered alike. More particularly, the collar 150 and retractor base 200 are present in the cranial access port 103.

The cranial access port 103 has an access port body 113 that is similar to the access port body 110 in that it includes the upper section 122, the lower section 124 and the bent section 120. The degree of bend in the access port body 113 can be the same as the degree of bend in the access port body 110. The access port body 110 is a hollow structure that defines a single lumen 119 that is open at the top end and the bottom end of the access port body 113.

In this embodiment, a flexible endoscope with working channels 300 is disposed within the single lumen 119. The flexible endoscope with working channels 300 has the same shape as the single lumen 119 and therefore, the flexible endoscope with working channels 300 has a bent construction defined by a linear upper section, a middle bent section and a linear lower section. As shown in FIGS. 15 and 16, the outer diameter of the flexible endoscope with working channels 300 is less than the inner diameter of the access port body 113 resulting in an open space (gap) between the flexible endoscope with working channels 300 and the access port body 113. As shown in the figures, the flexible endoscope with working channels 300 has a greater length than the access port body 113 which results in the top end of the flexible endoscope with working channels 300 being above the top end of the access port body 113 and the bottom end of the flexible endoscope with working channels 300 is located below the bottom end of the access port body 113.

The flexible endoscope with working channels 300 is a hollow (tubular) flexible structure that includes a suction (first) lumen 310 that extends from the top end to the bottom end and is open at both ends. As with the other embodiments, the suction lumen 310 is to be operatively coupled to a suction source so as to generate negative pressure within the suction lumen 310 for aspiration the combined irrigation fluid and the hematoma. The flexible endoscope with working channels 300 also includes a camera module 320. The camera module 320 is offset and isolated from the suction lumen 310 and in particular, is located radially outward from the suction lumen 310. The camera module 320 is thus located within a camera lumen that is open at both ends of the flexible endoscope with working channels 300 with the head of the camera module 320 being positioned at the bottom end of the flexible endoscope with working channels 300. The camera module 320 of the flexible endoscope with working channels 300 comprises the visualization device that is forward looking into the subdural space for visualization of the subdural hematoma. The camera module 320 can be in the form of a fiber optic cable.

As described herein, the cranial access ports disclosed herein provide a subdural hematoma evacuation solution that can be performed at bedside and in the angiography suite, thereby decreasing the number of anesthesia sessions necessary, which can be cumbersome for older patients. The present solution also provides active suction and a 360 degree view of the subdural space will provide the surgeon with the force necessary to remove viscous hematoma and the visual feedback to identify key structures and determine evacuation completion.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A cranial access port comprising:
a port body having a bend incorporated therein and having an ellipse cross-section, the port body being configured to be disposed at least partially within a subdural space and the port body having at least one lumen formed therein, the at least one lumen being configured to receive at least one tool for performing at least one operation within the subdural space.

2. The cranial access port of claim 1, wherein the at least one lumen comprises a first lumen and a second lumen and the at least one tool comprises an endoscope and at least one other tool, the first lumen being configured to receive the endoscope and the second lumen being configured to receive the at least one other tool.

3. The cranial access port of claim 2, wherein the first and second lumens are formed side-by-side.

4. The cranial access port of claim 1, wherein the ellipse cross-section is defined by a major axis that extends end-to-end of the port body and a minor axis that extends side-to-side of the port body and is oriented perpendicular to the major axis, the lengths of the major axis and the minor axis permitting the port body to be received within a 1 cm sized subdural space.

5. The cranial access port of claim 4, wherein the length of the major axis is at least 8 mm and the length of the minor axis is less than 8 mm.

6. The cranial access port of claim 5, wherein the length of the minor axis is from 4 mm to 6 mm.

7. The cranial access port of claim 1, wherein the port body has an upper section that is linear shaped and a lower section that is linear shaped with a bent section therebetween.

8. The cranial access port of claim 7, wherein the upper section is defined by a first axis that is perpendicular to the first end of the port body and the lower section is defined by a second axis that is perpendicular to the second end of the port body, the first and second axes being perpendicular to one another.

9. The cranial access port of claim 1, wherein the bend is defined by an angle greater than 0 to 180 degrees.

10. The cranial access port of claim 1, wherein the bend is defined by an angle greater than 0 to 90 degrees.

11. The cranial access port of claim 1, wherein the port body is formed of a rigid material and the bend comprises a fixed angle bend.

12. The cranial access port of claim 1, wherein the port body is formed of a flexible material that permits the bend to have a variable angle.

13. The cranial access port of claim 1, further comprising a collar that surrounds an upper section of the port body above the bend, the collar extending radially outward from the port body, wherein the collar has a prescribed height that prevents the cranial access port from falling into the brain.

14. The cranial access port of claim 13, wherein the collar has a circular shape with an ellipse shaped center hole through which the port body passes.

15. The cranial access port of claim 13, wherein the collar is coupled to the port body in a non-rotational manner or is integrally formed therewith to define a single part construction.

16. The cranial access port of claim 2, wherein the first lumen has a mirror located therein.

17. The cranial access port of claim 16, wherein the mirror is located within the bend of the port body and is positioned such that light traveling within a linear upper section of the port body is reflected by the mirror into a lower section of the port body that includes the bend.

18. The cranial access port of claim 17, wherein the mirror is disposed at a 45 degree angle within the first lumen so as to reflect light at an angle of 90 degrees.

19. The cranial access port of claim 13, wherein the collar is a separate part from the port body.

20. The cranial access port of claim 13, wherein the collar and the port body comprise a single part.

21. The cranial access port of claim 13, further comprising a stabilizing base configured to be fixedly attached to a patient's scalp or skull and includes an opening to permit the port body to pass therethrough.

22. The cranial access port of claim 21, wherein the stabilizing base includes a top section and an outwardly fared bottom section, the top section having an annular shape.

23. The cranial access port of claim 22, wherein the outwardly flared bottom section includes a plurality of notches formed therein, each notch being open along a perimeter edge of the outwardly flared bottom section, the plurality of notches being configured for receiving a scalp retractor tool.

24. The cranial access port of claim 23, wherein the plurality of notches comprises a first pair of notches located in a first half of the stabilizing base and a second pair of notches located in a second half of the stabilizing base.

25. The cranial access port of claim 22, wherein the top section of the stabilizing base is configured to be received internally within the collar resulting in the collar surrounding the top section and the collar and port body being rotatably coupled to the stabilizing base.

26. The cranial access port of claim 1, wherein the port body includes one or more visible markers to provide a user with information on rotation of the port body during a procedure.

27. The cranial access port of claim 26, wherein the information comprises degrees of rotation of the port body.

28. The cranial access port of claim 1, wherein the port body includes one or more radiopaque markers.

29. The cranial access port of claim 28, wherein the radiopaque markers are located at or proximate the second end of the port body.

30. The cranial access port of claim 21, wherein each of the collar and stabilizing base is located above the bend and each lies in a horizontal plane that is parallel to a plane that passes through a major axis of the second end of the port body.

31. The cranial access port of claim 2, further including a third lumen formed in the port body.

32. A multi-lumen cranial access device comprising:
an elbow shaped access port that has an ellipse cross-section, the access port having a first lumen and a second lumen formed therein, each of the first lumen and the second lumen being open at opposite first and second ends of the access port, the first lumen being configured to receive an endoscope and the second lumen being configured to receive at least one tool;
a collar that is non-rotatably coupled to the access port, the collar extending radially outward from the access port; and
a stabilizing base configured to be fixedly attached to a patient's scalp or skull, the stabilizing base includes an opening to permit the access port to pass therethrough, the stabilizing base including a top section that is rotatably coupled to the collar.

33. A subdural hematoma evacuation system comprising:
a rotatable elbow shaped access port that has an ellipse cross-section, the access port having at least one lumen formed therein, the first lumen being open at opposite first and second ends of the access port;
a visualization device received within the at least one lumen for providing visualization of a subdural space; and
at least one tool received within the at least one lumen for operating on and evacuating the subdural hematoma.

34. The system of claim 33, wherein the ellipse cross-section is defined by a major axis that extends end-to-end of the access port and a minor axis that extends side-to-side of the access port and is oriented perpendicular to the major axis, the lengths of the major axis and the minor axis permitting the port body to be received within a 1 cm subdural space.

35. The system of claim 34, wherein the length of the major axis is at least 8 mm and the length of the minor axis is less than 8 mm.

36. The system of claim 35, wherein the length of the minor axis is from 4 mm to 6 mm.

37. The system of claim 33, wherein the elbow shaped access port comprises a 90 degree elbow.

38. The system of claim 33, wherein the elbow shaped access portion is defined by bend that is defined by an angle greater than 0 to 180 degrees.

39. The system of claim 38, wherein the bend is defined by an angle greater than 0 to 90 degrees.

40. The system of claim 3, wherein the access port is formed of a rigid material and includes a fixed angle bend.

41. The system of claim 33, wherein the access port is formed of a flexible material that permits the bend to have a variable angle.

42. The system of claim 33, further comprising a collar that surrounds an upper section of the access port above a bend, the collar extending radially outward from the port body, wherein the collar has a prescribed height that prevents the cranial access port from freely falling into the brain.

43. The system of claim 42, wherein the collar has a circular shape with an ellipse shaped center hole through which the access port passes.

44. The system of claim 42, wherein the collar is coupled to the access port in a non-rotational manner.

45. The system of claim 33, wherein the access port is configured such that an upper section thereof can be oriented perpendicular to a burr hole through a patient's skull, while a lower section thereof is oriented at a 90 degree angle relative to the upper section to allow a user to evacuate the subdural hematoma.

46. The system of claim 33, wherein the visualization device comprises a rigid endoscope.

47. The system of claim 46, wherein the rigid endoscope is coupled to the access port such that a distal end of the rigid endoscope is disposed within a linear upper section of the access port that is located above a bend in the access port.

48. The system of claim 47, further including a mirror located in the bend below the distal end of the rigid endoscope, the mirror being positioned such that light traveling within the linear upper section of the access port is reflected by the mirror into a lower section of the access port below the bend.

49. The system of claim 48, wherein the mirror is disposed at a 45 degree angle within the first lumen so as to reflect light at an angle of 90 degrees.

50. The system of claim 33, further comprising a stabilizing base configured to be fixedly attached to a patient's scalp or skull and includes an opening to permit the access port to pass therethrough.

51. The system of claim 50, wherein the stabilizing base includes a top section and an outwardly fared bottom section, the top section having an annular shape.

52. The system of claim 51, wherein the outwardly flared bottom section includes a plurality of notches formed therein, each notch being open along a perimeter edge of the outwardly flared bottom section, the plurality of notches being configured for receiving a scalp anchoring tool.

53. The system of claim 52, wherein the plurality of notches comprises a first pair of notches located in a first half of the stabilizing base and a second pair of notches located in a second half of the stabilizing base.

54. The system of claim 51, further comprising a collar that surrounds an upper section of the access port above a bend, the collar extending radially outward from the port body and wherein the top section of the stabilizing base is configured to be received internally within the collar resulting in the collar surrounding the top section and the collar and port body being rotatably coupled to the stabilizing base.

55. The system of claim 33, wherein the access port includes one or more visible markers to provide a user with information on rotation of the port body during a procedure.

56. The system of claim 55, wherein the information comprises degrees of rotation of the port body.

57. The system of claim 33, wherein the access port includes one or more radiopaque markers.

58. The system of claim 33, wherein the visualization device comprises a flexible endoscope that is capable of exiting the second end of the access port.

59. The system of claim 33, wherein the visualization device docks with the access port.

60. The system of claim 33, wherein the visualization device comprises a rigid endoscope and one of the rigid endoscope and the access port includes a stop that limits a degree of travel of the rigid endoscope within the first lumen.

61. The system of claim 33, wherein the at least one tool comprises a suction and irrigation device.

62. The system of claim 33, further including a steering mechanism that enables 360 degree rotation of the access port in the subdural space.

63. The system of claim 33, further including a video display operatively coupled to the visualization device.

64. The system of claim 33, wherein the at least one tool comprises at least one tool selected from the group consisting of: an irrigation tool, an aspiration tool, a coagulation tool, and a tissue grasping tool.

65. The system of claim 33, wherein the at least one tool comprises a catheter that is insertable in the second lumen and includes an irrigation lumen and/or an aspiration lumen.

66. The system of claim 33, wherein the access port is configured to be received within and pass through a burr hole having a diameter from 8 mm to 14 mm.

67. An subdural hematoma evacuation system comprising:
a rotatable elbow shaped access port that has an ellipse cross-section along a length thereof, the access port being defined by a first leg and a second leg with a bend therebetween, the access port having a first lumen and a second lumen formed therein;
a stabilizer that is configured to be fixedly attached to a patient's scalp or skull and includes an opening to permit the access port to pass therethrough and permit 360 degrees of rotation of the access port relative to the stabilizer; and
a visualization device received within the first lumen and at least one optical element contained within the first lumen and within the bend of the access port for providing visualization of an subdural space from the second leg.

68. The system of claim 67, wherein an angle between the first leg and the second leg is greater than 0 but less than 180 degrees.

69. The system of claim 68, wherein the angle is 90 degrees.

70. The system of claim 67, wherein the visualization device is docked within the first lumen such that the visualization device does not rotate within the first lumen.

71. A method for evacuating a subdural hematoma comprising the steps of:
forming a burr hole through a patient's skull;
inserting an elbow shaped access port through the burr hole and at least partially within a subdural space, the access port having an ellipse cross-section, the access port having a first lumen and a second lumen formed therein;
inserting a visualization device into the first lumen; and
obtaining an image of the subdural space with the visualization device to locate and observe the subdural hematoma; and
using at least one tool that is inserted through the second lumen to remove the subdural hematoma under guidance from the visualization device.

72. The method of claim 71, further including the step of stabilizing the access port on the skull with a stabilizing base that is fixed to the skull but permits rotation of the access port relative to the stabilizing base to permit access port to be rotated 360 degrees within the subdural space.

73. The method of claim 71, wherein the visualization device comprises a rigid endoscope that is inserted into the first lumen and is docked to the access port such that a distal end of the rigid endoscope is disposed within a linear upper section of the access port that is located above a bend in the access port and the access port further includes a mirror located in the bend below the distal end of the rigid endoscope, the mirror being positioned such that light traveling within the linear upper section of the access port is reflected by the mirror into a lower section of the access port below the bend.

74. The method of claim 71, wherein the step of using at least one tool comprises inserting a catheter into and through the second lumen to the subdural space.

75. The method of claim 71, wherein the step of removing the subdural hematoma comprises applying an irrigation fluid to the subdural space and aspirating the subdural hematoma.

76. The method of claim 71, wherein the visualization device comprises a flexible endoscope that travels beyond a bend in the access port for viewing the subdural space.
